(19) United States
(12) Patent Application Publication
(10) Pub. No.: US 2023/0134630 A1
(43) Pub. Date: May 4, 2023

(54) INFERENCE APPARATUS, MEDICAL IMAGE DIAGNOSTIC APPARATUS, INFERENCE METHOD, AND TRAINED NEURAL NETWORK GENERATION METHOD

(71) Applicant: Canon Medical Systems Corporation, Otawara-shi (JP)

(72) Inventor: Hidenori TAKESHIMA, Tokyo (JP)

(73) Assignee: Canon Medical Systems Corporation, Otawara-shi (JP)

(21) Appl. No.: 18/051,081

(22) Filed: Oct. 31, 2022

(30) Foreign Application Priority Data
Nov. 1, 2021 (JP) 2021-178506

(51) Int. Cl. G06N 3/08; G06N 5/04

(52) U.S. Cl. CPC G06N 3/08; G06N 5/04

(57) ABSTRACT

According to one embodiment, an inference apparatus includes a processing circuit configured to: obtain processing target data; and calculate inference data by applying a trained neural network to the processing target data, wherein the trained neural network includes an ensemble activation function for each of a plurality of unit network structures configured to convert an input vector element to an output vector element, the ensemble activation function being configured to execute a calculation based on a plurality of activation functions and a plurality of mixing coefficients respectively corresponding to the activation functions.

### CROSS-REFERENCE TO RELATED APPLICATIONS

This application is based upon and claims the benefit of priority from Japanese Patent Application No. 2021-178506, filed Nov. 1, 2021, the entire contents of which are incorporated herein by reference.

### FIELD

Embodiments described herein relate generally to an inference apparatus, a medical image diagnostic apparatus, an inference method, and a trained neural network generation method.

### BACKGROUND

For a neural network configured to execute a desired inference upon medical data including medical image data or its raw data, a deep neural network (DNN) or a convolutional neural network (CNN) may be adopted.

For such a neural network, various types of activation functions have been discussed as functions to be used for activation. Examples of such activation functions may include a logistic sigmoid function (logistic function), a hyperbolic tangent function (tanh), a rectified liner unit (ReLU), linear mapping, identity mapping, a maxout function, an ELU, a Leaky ReLU, and a complex ReLU. Whichever activation function is adopted, the inference performance of the function demonstrates advantages and disadvantages.

In the conventional technique, the type of activation function to be used for a neural network is determined by a designer of the network. The designer therefore needs to find the activation function that best suits the purpose of use and optimization approach. It has been known, however, that the selection of an activation function type has a significant influence upon the inference performance of a neural network. For this reason, there has been a demand for suitable selection of an activation function that can improve the inference performance of the neural network.

### BRIEF DESCRIPTION OF THE DRAWINGS

FIG. 1 is a diagram showing an overview of the configuration and processing of a medical data processing system, in which a medical data processing apparatus according to the present embodiment is incorporated.

FIG. 2 is a diagram showing the configuration of a neural network according to the present embodiment.

FIG. 3 is a diagram schematically showing a calculation performed by an ensemble activation function according to the present embodiment.

FIG. 4 is a diagram showing the configuration of convolution layers according to the present embodiment.

FIG. 5 is a diagram showing the configuration of a medical image diagnostic apparatus according to the present embodiment.

FIG. 6 is a diagram showing an exemplary combination of an input and an output of a trained neural network according to the present embodiment. FIG. 7 is a diagram showing the configuration of a model training apparatus according to the present embodiment.

FIG. 8 is a diagram schematically showing the calculation of an ensemble activation function according to the first modification example of the present embodiment.

FIG. 9 is a diagram schematically showing the calculation of an ensemble activation function according to the second modification example of the present embodiment.

### DETAILED DESCRIPTION

In general, according to one embodiment, an inference apparatus includes a processing circuit configured to: obtain processing target data; and calculate inference data by applying a trained neural network to the processing target data, wherein the trained neural network includes an ensemble activation function for each of a plurality of unit network structures configured to convert an input vector element to an output vector element, the ensemble activation function being configured to execute a calculation based on a plurality of activation functions and a plurality of mixing coefficients respectively corresponding to the activation functions.

The embodiments of an inference apparatus and a trained neural network generation method will be described in detail below, with reference to the drawings. The inference apparatus is configured to find inference data by applying a trained neural network to the processing target data. A typical inference apparatus is a medical data processing apparatus that receives the processing-target input medical data as an input and outputs corresponding output medical data. The input medical data is the processing target data. The inference apparatus may not be limited to an apparatus for use in the medical field, but may be used in inference processing for data other than medical data. In this embodiment, an inference apparatus executing processing upon medical data will be discussed. In the following description, structural elements having substantially the same operations and configurations will be denoted by the same reference symbols, and the same explanation will be given only where necessary.

### (Embodiment)

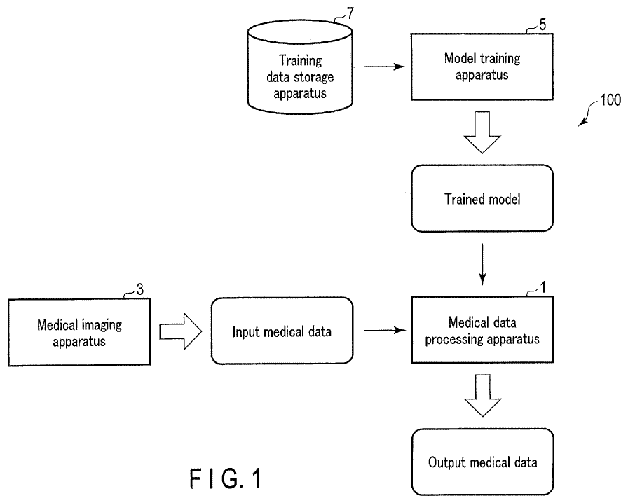

FIG. 1 is a diagram showing an overview of the configuration and processing of a medical data processing system 100 that includes a medical data processing apparatus 1, which serves as an inference apparatus according to the present embodiment. As illustrated in FIG. 1, the medical data processing system 100 according to the present embodiment includes a medical data processing apparatus 1, a medical imaging apparatus 3, a model training apparatus 5, and a training data storage apparatus 7.

The training data storage apparatus 7 stores training data that includes a plurality of training samples. For instance, the training data storage apparatus 7 may be a computer provided with a storage device therein. Alternatively, the training data storage apparatus 7 may be a mass storage device connected to a computer in a communicable manner via a cable or a communication network. For such a storage device, a hard disk drive (HDD), a solid state drive (SSD), and an integrated circuit memory device may be adopted as appropriate.

The model training apparatus 5 performs machine training upon machine learning models in accordance with a model training program, based on the training data stored in the training data storage apparatus 7, thereby generating a trained machine learning model (hereinafter referred to as a “trained model”). The model training apparatus 5 is a computer, such as a workstation, having a processor such as a central processing unit (CPU) and a graphics processing unit (GPU). The model training apparatus 5 and the training data storage apparatus 7 may be connected to each other in a communicable manner via a cable or a communication network. Alternatively, the training data storage apparatus 7 may be mounted on the model training apparatus 5. If this is the case, the training data is supplied from the training data storage apparatus 7 to the model training apparatus 5 via a cable, a communication network, or the like. The model training apparatus 5 and the training data storage apparatus 7 may not be connected to each other in a communicable manner. If this is the case, the training data is supplied from the training data storage apparatus 7 to the model training apparatus 5 by way of a portable storage medium or the like that stores the training data. The model training apparatus 5 is an exemplary learning apparatus.

The machine learning model according to the present embodiment is a parameter-added composite function obtained by combining a plurality of functions. A parameter-added composite function is defined by a combination of multiple adjustable functions and parameters. The machine learning model according to the present embodiment may be any parameter-added composite function that satisfies the above requirements as long as it is a multi-layered network model (hereinafter referred to as a “multi-layered network”). As a multi-layered network, a neural network such as a deep neural network (DNN) or a convolutional neural network (CNN) having convolution layers may be used. In the following description, a neural network is adopted as a multi-layered network according to the present embodiment. The neural network according to the present embodiment receives, as an input, processing-target input medical data obtained by the medical imaging apparatus 3, and outputs the corresponding output medical data.

The medical imaging apparatus 3 generates processing-target medical data. Conceptually, the medical data according to the present embodiment includes raw data collected by the medical imaging apparatus 3 or some other medical imaging apparatus that performs medical imaging on a subject, and medical image data generated by performing restoration processing upon this raw data. The medical imaging apparatus 3 can be an apparatus of any modality as long as it is capable of generating medical data. For instance, the medical imaging apparatus 3 according to the present embodiment may be a single modality apparatus such as a magnetic resonance imaging (MRI) apparatus, an X-ray computed tomography imaging (CT) apparatus, an X-ray diagnostic apparatus, a positron emission tomography (PET) apparatus, a single photon emission CT (SPECT) apparatus, or an ultrasonic diagnostic apparatus. Alternatively, it may be a combined modality apparatus such as a PET/CT apparatus, SPECT/CT apparatus, PET/MRI apparatus, or SPECT/MRI apparatus.

The medical data processing apparatus 1 generates output medical data corresponding to the processing-target input medical data obtained by the medical imaging apparatus 3, using a trained model, which has been trained by the model training apparatus 5 in accordance with a model training program. The medical data processing apparatus 1 and the model training apparatus 5 may be connected to each other in a communicable manner via a cable or a communication network. Alternatively, the medical data processing apparatus 1 and the model training apparatus 5 may be mounted on the same computer. If this is the case, a trained model is supplied from the model training apparatus 5 to the medical data processing apparatus 1 via a cable, a communication network, or the like. The medical data processing apparatus 1 and the model training apparatus 5 may not always be connected to each other in a communicable manner. If this is the case, a trained model is supplied from the model training apparatus 5 to the medical data processing apparatus 1 by way of a portable storage medium or the like that stores the trained model. The trained model may be supplied at any time, i.e., at a time between the manufacture of the medical data processing apparatus 1 and installation at a medical facility or the like, or at the time of maintenance. The supplied trained model is stored in the medical data processing apparatus 1. Furthermore, the medical data processing apparatus 1 may be a computer mounted on a medical image diagnostic apparatus that includes the medical imaging apparatus 3. Alternatively, it may be a computer connected to such a medical image diagnostic apparatus via a cable or network in a communicable manner, or it may be a computer provided independently from the medical image diagnostic apparatus.

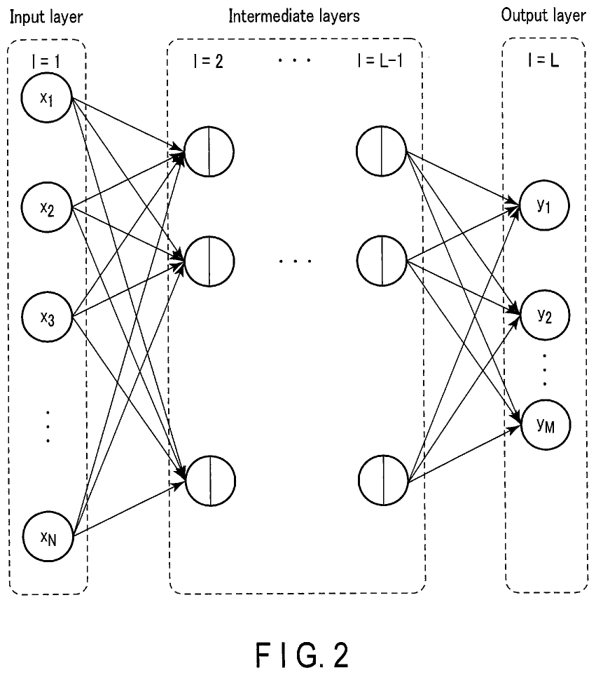

A typical configuration of a neural network according to the present embodiment will be discussed. FIG. 2 is a diagram showing the typical configuration of a neural network according to the present embodiment. A neural network here means a network having a layered structure in which only adjacent layers are coupled to each other so that information propagates in one direction from the input layer side to the output layer side. The neural network according to the present embodiment is a feedforward network, in which image data input to the input layer propagates from the input layer side toward the output layer side, with only adjacent layers being coupled to each other.

As illustrated in FIG. 2, the neural network according to the present embodiment is constituted by the number L of layers, namely, an input layer (l=1), intermediate layers (l=2, 3, . . . , L−1), and an output layer (l=L). An exemplary neural network is described below, although the configuration of the neural network should not be limited thereto.

Input data is input to the input layer (first layer). The input data may be image data including medical image data, or raw data including k-space data and projection data. In the input layer, the input data becomes output data as is.

In the intermediate layers (l=2, 3, . . . , L−1) that follow the input layer, calculations are subsequently executed based on weighting matrices between the layers, a bias of each layer, and activation of each layer so that the calculated values are output.

In the output layer (Lth layer) that follows the intermediate layers, the data input from the intermediate layers becomes output data as is.

The neural network according to the present embodiment is a feedforward network, in which the data input to the input layer propagates from the input layer side toward the output layer side, with only adjacent layers being coupled to each other. Such a feedforward network is defined as a composite function that is a combination of a linear correlation between the layers using a weighting matrix W, a nonlinear correlation (or linear correlation) using activation of each layer, and a bias. The weighting matrix and bias are referred to as parameters p of the network. The form of the composite function thus defined varies, depending on the selection of the parameters p. This means that the neural network according to the present embodiment can be defined as a function capable of outputting a suitable result from the output layer by suitably selecting the parameters p of the composite function.

A neural network is constituted by a plurality of unit network structures. A unit network structure is a unit of elements that constitute the network, and each unit network structure converts an input vector element to an output vector element. A unit network structure may correspond to a node, a layer, a channel, or a unit. Each unit network structure may receive, as an input, an input vector element that includes an output value of a different unit network structure, and calculate values by adding a bias b to respective products of the values of the input vector element and different weights, thereby executing activation processing upon the calculated values.

In the activation processing, a single value obtained by executing a nonlinear conversion (or linear conversion) upon the calculated values is obtained. Then, the calculated value is output as an output of this unit network structure.

The present embodiment adopts an ensemble activation function, which is a novel function, in the activation processing. To generate an ensemble activation function, a plurality of activation functions are prepared, and training is performed by incorporating portions of the activation functions that are to be integrated, into a training process. More specifically, in the ensemble activation function, different activation functions are applied to the same data, and a data calculation is performed in accordance with the activation functions and mixing coefficients to output an output value.

For instance, the ensemble activation function applies different activation functions to the same data, integrates the output values of the activation functions in accordance with the mixing coefficients, and outputs the integrated output value.

As activation functions, a logistic sigmoid function (logistic function), a hyperbolic tangent function (tanh), a rectified liner unit (ReLU), linear mapping, identity mapping, a maxout function, and the like may be selected in accordance with the purpose. Alternatively, an ELU, a Leaky ReLU, a Complex ReLU, and the like may be adopted as activation functions.

A mixing coefficient is a weighted parameter provided for each of the activation functions that are to be integrated. The mixing coefficient is determined, for example, through optimization together with other weighted parameters included in the neural network in the process of training the neural network so that the desired output data can be output in response to an input of certain input data.

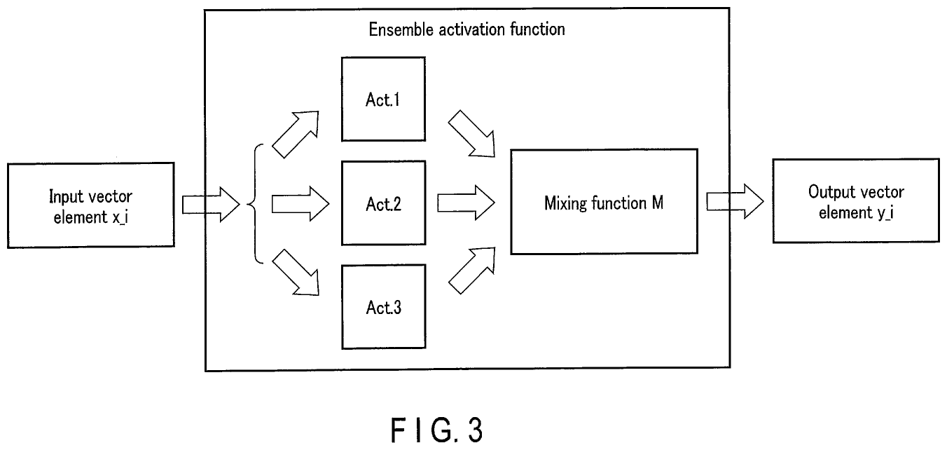

An ensemble activation function according to the present embodiment will be explained below with reference to FIG. 3, which is a diagram schematically showing the calculation performed by an ensemble activation function according to the present embodiment. As illustrated in FIG. 3, the ensemble activation function is constituted by three activation functions A1 to A3 and a mixing function M. The activation functions A1 to A3 are activation functions of different types. In FIG. 3, the activation functions A1 to A3 are illustrated as “Act.1” to “Act.3”. The number of activation functions in the ensemble activation function may be two, or may be four or more.

The ensemble activation function receives an input vector element x_i. The input vector element x_i denotes a vector constituted by a plurality of input values x that have been output through a certain channel i that constitutes, for example, a CNN.

The input vector element x_i is inserted into each of the activation functions A1 to A3. The activation function A1 performs a calculation using the values x of the input vector element x_i as an input, and outputs the result of the calculation as an output value z1. Similarly, the activation functions A2 and A3 respectively perform a calculation using the values x of the input vector element x_i as an input, and output the results of the calculation as output values z2 and z3.

The mixing function M receives, as inputs, the output values of the activation functions of different preset types, and outputs a single output value obtained by integrating the received output values. In the example of FIG. 3, the mixing function M integrates, in accordance with a mixing coefficient, the three output values z1 to z3 received from the activation functions A1 to A3 to find an output value y.

The mixing function M may be a function for calculating a linear sum of weighted input values or a nonlinear sum of the weighted input values. The mixing function M may be a linear function or a quadratic function receiving multiple inputs that are variables, or a function in the form of a polynomial having a dimensionality of 3 or larger. Alternatively, a kernel function used for the kernel trick may be adopted as a mixing function M, or a neural network expressed as a parameter-added composite function of multiple functions may be adopted as a mixing function M.

For instance, if an ensemble activation function is formed of two types of activation functions, the mixing function M integrates the output values z1 and z2 of the two activation functions A1 and A2 of the different types, and calculates the output value y. Expression (1) indicates an exemplary computational formula for an output value y when a linear function is adopted as the mixing function M. In Expression (1), the coefficients a1 to a3 represent mixing coefficients according to the present embodiment. The coefficients a1 to a3 are determined in the process of optimizing the entire neural network, in which the optimization of the coefficients a1 to a3 is performed together with other parameters. According to Expression (1), the mixing function M calculates a value y by multiplying the output values z1 and z2 of the activation functions A1 and A2 by different coefficients and adding the multiplied values.

z=a1z1+a2z2+a3 (1)

Expression (2) is an exemplary computational formula for an output value y when a quadratic function is adopted as the mixing function M. In Expression (2), the coefficients a1 to a6 are mixing coefficients according to the present embodiment. The coefficients a1 to a6 are determined in the process of optimizing the entire neural network, in which the optimization of the coefficients a1 to a6 is performed together with other parameters.

y=a1z1²+a2z1z2+a3z2²+a4z1+a5z2+a6 (2)

The above activation functions and mixing function M are sequentially applied to the values x included in the input vector element x_i, so that output values y corresponding to the values x of the input vector element x i are output. The ensemble activation function generates an output vector element y_i including the output values y that have been output to correspond to the values x of the input vector element x_i, and outputs the generated output vector element y_i as an output of the ensemble activation function.

Next, the use of a CNN as an example of the neural network will be explained. The CNN includes an input layer, convolution layers, a pooling layer, a fully connected layer, and an output layer. In the CNN, a plurality of processing blocks, for example in each of which a pooling layer is arranged after two convolution layers, are arranged in front of the fully connected layer. The number of convolution layers and pooling layers to be connected and the order of the connection may be suitably determined.

Input data is supplied to the input layer. Typically, the input data is vector data. The input data may be read out for each chunk of input data (e.g., for each channel) on the memory, or for each element including input data of multiple channels. In a convolution layer, convolution processing is executed upon the input data from the input layer. In a pooling layer, max pooling processing is executed upon the data that has been subjected to the convolution processing. In the fully connected layer, the data processed in the processing blocks and the channels of the fully connected layer are fully coupled to each other. In the output layer, output data is generated as the final output from the CNN.

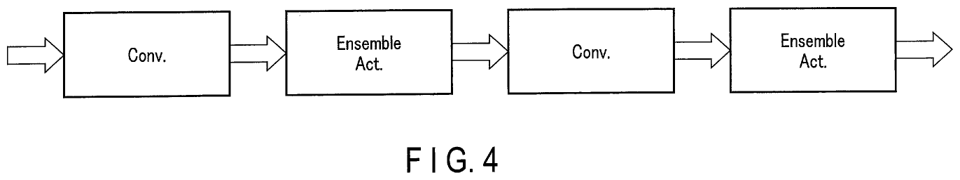

In the convolution layers, convolution, regularization, and activation processing are executed. The regularization and activation processing are not essential, and may not always need to be executed. If the activation processing is not performed in the convolution layers, this processing is performed in a layer other than the convolution layers. FIG. 4 is a diagram schematically showing a typical configuration of the convolution layers. In FIG. 4, convolution processing is indicated as “Conv.”, and activation processing is indicated as “ensemble Act.” The regularization is omitted from FIG. 4.

In the convolution processing, the processing is executed upon the input data of the input layer for each channel. For instance, the convolution processing is performed using one kernel (filter) for each channel in the convolution processing, and the data subjected to the convolution processing is produced as a feature map.

In the regularization processing, the feature map subjected to the convolution processing is input, and the regularization is executed upon this feature map. For instance, batch normalization and dropout may be employed as the regularization. A commonly known process can be adopted for the regularization.

In the activation processing, the aforementioned ensemble activation function is applied to the data subjected to the convolution or regularization to generate the final output data of the convolution layer. The output data is input to a convolution layer or pooling layer provided adjacent to the convolution layer and downstream thereof.

In the output layer, activation processing is executed upon the output of the fully connected layer, using an activation function. In the activation processing, a softmax function may be applied as an activation function. Alternatively, some other activation function that corresponds to a desired output format may be applied.

For example, if the CNN is used for binary classification, a logistic function is adopted as an activation function.

If the CNN is used for regression, linear mapping is adopted as an activation function.

The aforementioned ensemble activation function may be used for the activation processing in the output layer instead of the activation processing in the convolution layer. The ensemble activation function may be used for the activation processing in both the convolution layer and output layer.

An exemplary configuration of the medical data processing apparatus 1 according to the present embodiment will be described below. It is assumed here that the medical data processing apparatus 1 is coupled to the medical imaging apparatus 3, and is incorporated together with the medical imaging apparatus 3 into a medical image diagnostic apparatus 9.

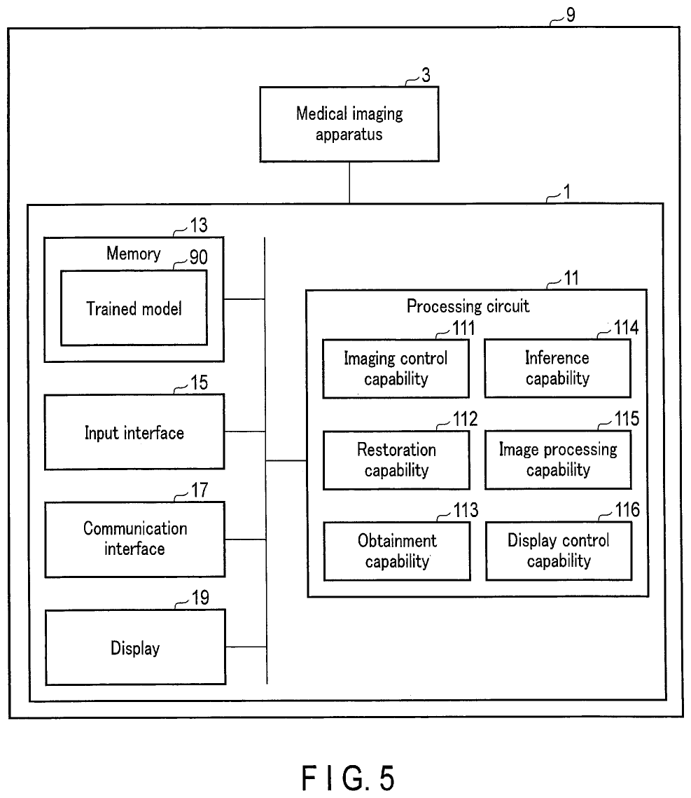

FIG. 5 is a diagram showing the configuration of the medical image diagnostic apparatus 9 according to the present embodiment. As illustrated in FIG. 5, the medical image diagnostic apparatus 9 includes the medical data processing apparatus 1 and the medical imaging apparatus 3.

In one example, the medical imaging apparatus 3 corresponds to a gantry, and the medical data processing apparatus 1 corresponds to a console connected to the gantry. The medical data processing apparatus 1 may be arranged on the gantry of the medical image diagnostic apparatus 9, or may be realized by a component other than the console or gantry of the medical image diagnostic apparatus 9. For such a component, a computer other than the console or a dedicated computing machine may be installed in a machine room if the medical image diagnostic apparatus 9 is a magnetic resonance imaging apparatus.

The medical imaging apparatus 3 provides medical imaging upon a subject on the imaging principles corresponding to the modality type of the medical imaging apparatus 3, and collects raw data of the subject. The collected raw data is transferred to the medical data processing apparatus 1. For instance, the raw data is k-space data if the medical imaging apparatus 3 is a magnetic resonance imaging apparatus, projection data or sinogram data in the case of an X-ray computed tomography imaging apparatus, echo data in the case of an ultrasonic diagnostic apparatus, coincidence data or sinogram data in the case of a PET apparatus, and projection data or sinogram data in the case of a SPECT apparatus. If the medical imaging apparatus 3 is an X-ray diagnostic apparatus, the raw data is X-ray image data. The medical imaging apparatus 3 is an example of an imaging unit.

If the medical imaging apparatus 3 is a gantry for a magnetic resonance imaging apparatus, this gantry repeats application of a gradient magnetic field by way of a gradient magnetic field coil and application of RF pulses by way of a transmission coil under the application of a static magnetic field by way of a static magnetic field magnet. In response to the application of the RF pulses, an MR signal is emitted from the subject. The emitted MR signal is received via the reception coil. The received MR signal is subjected to signal processing such as A/D conversion by the reception circuit. The A/D-converted MR signal is referred to as k-space data. The k-space data is transferred as raw data to the medical data processing apparatus 1.

If the medical imaging apparatus 3 is a gantry for an X-ray computed tomography imaging apparatus, the gantry applies X-rays to the subject from the X-ray tube while rotating the X-ray tube and X-ray detector around the subject, and detects the X-rays that have passed through the subject with the X-ray detector. In the X-ray detector, an electric signal having a crest value corresponding to the detected X-ray dose is generated. This electric signal is subjected to signal processing such as A/D conversion by a data collection circuit. The A/D-converted electrical signal is referred to as projection data or sinogram data. The projection data or sinogram data is transferred as raw data to the medical data processing apparatus 1.

If the medical imaging apparatus 3 is an ultrasonic probe of an ultrasonic diagnostic apparatus, the ultrasonic probe transmits ultrasonic beams from a plurality of ultrasonic transducers to the inside of the body of the subject, and receives ultrasonic waves reflected from the inside of the body by way of the ultrasonic transducers. The ultrasonic transducers generate electric signals having a crest value corresponding to the sound pressure of the received ultrasonic waves. The electric signals are subjected to A/D conversion by an A/D converter provided in the ultrasonic probe or the like. The A/D-converted electric signals are referred to as “echo data”. The echo data is transferred as raw data to the medical data processing apparatus 1.

If the medical imaging apparatus 3 is a gantry for a PET apparatus, a simultaneous measurement circuit simultaneously measures a pair of 511 keV gamma rays generated in accordance with the pair annihilation of positrons generated from radionuclides accumulated in the subject body and electrons around the radionuclides so that the gantry can produce digital data having a digital value indicative of the energy value and detection position of the pair of gamma rays (line of response, or LOR). This digital data is referred to as “coincidence data” or “sinogram data”. The coincidence data or sinogram data is transferred as raw data to the medical data processing apparatus 1.

If the medical imaging apparatus 3 is a C-arm of an X-ray diagnostic apparatus, the X-ray is generated from an X-ray tube provided in the C-arm. An X-ray detector such as a flat panel detector (FPD) arranged in the C-arm or independently from the C-arm receives the X-ray that has been generated from the X-ray tube and transmitted through the body of the subject. The X-ray detector generates an electric signal having a crest value that corresponds to the detected X-ray dose, and executes signal processing such as A/D conversion upon the electric signal. The A/D-converted electric signal is referred to as “X-ray image data”. The X-ray image data is transferred as raw data to the medical data processing apparatus 1.

As illustrated in FIG. 5, the medical data processing apparatus 1 includes, as hardware resources, a processing circuit 11, a memory 13, an input interface 15, a communication interface 17, and a display 19.

The memory 13 is a storage device configured to store various types of information, such as a hard disk drive (HDD), a solid state drive (SSD), and an integrated circuit. In addition to an HDD and SSD, the memory 13 may be a portable storage medium such as a compact disc (CD), a digital versatile disc (DVD), and a flash memory. The memory 13 may be a driving device for reading and writing various types of information from and to a semiconductor memory element such as a flash memory and a random access memory (RAM). The storage region of the memory 13 may be provided in the medical data processing apparatus 1, or in an external storage device connected by way of a network.

The memory 13 is configured to store programs to be executed by the processing circuit 11, various types of data to be used in the processing performed by the processing circuit 11, and the like. Such programs may include a program that is installed in advance in a computer through a network or from a non-transitory computer-readable storage medium to cause the computer to realize various functions of the processing circuit 11. The typical data discussed throughout this specification is digital data. The memory 13 is an example of a storage unit.

The memory 13 is configured to store a trained model 90 generated by the model training apparatus 5. The trained model 90 is a neural network having a parameter p that has been trained so as to output targeted output medical data in response to an input of input medical data. The trained model 90 is an example of a trained neural network.

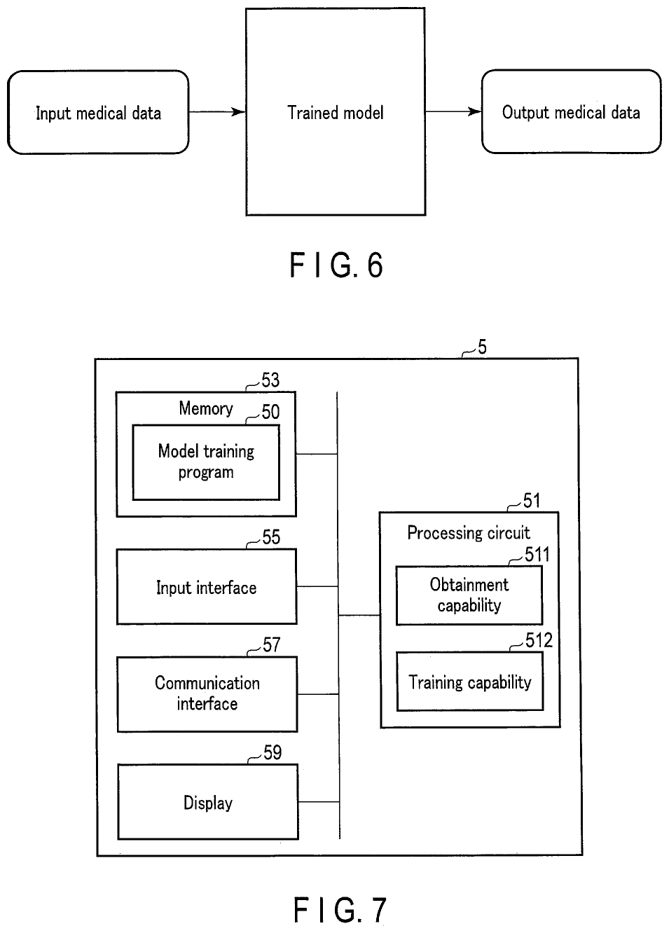

FIG. 6 is a diagram showing an exemplary combination of an input and an output of the trained model 90. The trained model 90 receives an input of the input data, and outputs the output data. For instance, as illustrated in FIG. 6, the trained model 90 receives input medical data, and outputs output medical data. The input medical data and output medical data may be medical image data relating to a processing-target subject. In place of the medical image data, raw data of the processing target may be used as input medical data and output medical data. This raw data relates to the processing-target subject. As input medical data, an input of a medical image may be received, and the result of identification of the medical image may be output as output medical data.

The raw data according to the present embodiment is not limited to the original raw data collected by the medical imaging apparatus 3. For instance, the raw data according to the present embodiment may be computational raw data generated by executing the forward projection processing upon a medical image generated by the restoration capability 112 or inference capability 114.

The raw data according to the present embodiment may be original raw data that has been subjected to any data processing such as data compression processing, resolution resolving processing, data interpolation processing, and resolution synthesizing processing. The raw data according to the present embodiment may be, if it is three-dimensional raw data, hybrid data subjected to the restoration processing for one or two axes only. Similarly, a medical image according to the present embodiment is not limited to an original medical image generated by the restoration capability 112 or inference capability 114. The medical image according to the present embodiment may be an original medical image that has been subjected to any image processing, such as image compression processing, resolution decomposition processing, image interpolation processing, and resolution combination processing.

The input interface 15 receives various input operations from an operator, converts the received input operation to electric signals, and outputs the signals to the processing circuit 11. For instance, the input interface 15 receives an input of medical information, an input of various command signals, and the like from the operator. The input interface 15 is realized by a mouse, a keyboard, a trackball, switch buttons, a touch screen in which a display screen and a touch pad are integrated, a non-contact input circuit adopting optical sensors, a voice input circuit, and the like for performing various processes of the processing circuit 11. The input interface 15 is connected to the processing circuit 11 so that the input operation received from the operator can be converted to an electric signal and can be output to the control circuit. Throughout this specification, the input interface is not limited to a physical operation component such as a mouse and a keyboard. Examples of the input interface may include an electric signal processing circuit configured to receive an electric signal corresponding to an input operation from an external input device provided separately from the apparatus, and output this electric signal to the processing circuit 11. The input interface 15 is an example of an input unit.

The communication interface 17 is a network interface configured to control the communication between the medical data processing apparatus 1 and an external apparatus via a network.

The display 19 is configured to display various types of information. For instance, the display 19 outputs medical information generated by the processing circuit 11, a graphical user interface (GUI) for receiving various operations from the operator, and the like. The display 19 may be a liquid crystal display or a cathode-ray tube (CRT) display. The display 19 is an example of a display unit.

The processing circuit 11 is configured to control the overall operation of the medical data processing apparatus 1. The processing circuit 11 is a processor configured to, upon calling up and executing a program from the memory 13, realize an imaging control capability 111, a restoration capability 112, an obtainment capability 113, an inference capability 114, an image processing capability 115, and a display control capability 116. FIG. 5 illustrates a single processing circuit 11 realizing the imaging control capability 111, restoration capability 112, obtainment capability 113, inference capability 114, image processing capability 115, and display control capability 116. These capabilities, however, may be realized by combining multiple independent processors to form a processing circuit, causing these processors to execute the program. The imaging control capability 111, restoration capability 112, obtainment capability 113, inference capability 114, image processing capability 115, and display control capability 116 may be implemented as individual hardware circuits. The above description of the capabilities implemented by the processing circuit 11 applies to the embodiments and modification examples described below.

In the description, the medical data processing apparatus 1 executes multiple capabilities with a single console. These capabilities, however, may be implemented by different devices. For instance, the capabilities of the processing circuit 11 may be distributed over different devices.

The term “processor” that appears in the above description denotes a central processing unit (CPU), a graphics processing unit (GPU), or a circuit such as ASIC, a programmable logic device (e.g., simple programmable logic device (SPLD) or complex programmable logic device (CPLD)), and a field programmable gate array (FPGA). The processor realizes the capabilities by reading and executing a program stored in the memory 13. Instead of storing programs in the memory 13, the programs may be directly incorporated into the circuit of the processor.

If this is the case, the processor realizes capabilities by reading and executing a program incorporated in the processor. The processors according to the embodiments are not limited to a single circuit for each processor, but may be configured as a single processor by combining different independent circuits to realize the capabilities. Furthermore, the structural components illustrated in FIG. 5 may be integrated into one processor to realize their capabilities. The above description of the “processor” applies to the embodiments and modification examples discussed below.

With the imaging control capability 111, the processing circuit 11 controls the medical imaging apparatus 3 in accordance with imaging conditions, and performs medical imaging upon the subject. The imaging conditions according to the present embodiment include imaging principles of the medical imaging apparatus 3 and various imaging parameters. The imaging principles correspond to the type of the medical imaging apparatus 3, or more specifically, to a magnetic resonance imaging apparatus, an X-ray computed tomography imaging apparatus, a PET apparatus, a SPECT apparatus, or an ultrasonic diagnostic apparatus. The imaging parameters may include a field of view (FOV), an imaging site, a slice position, a frame (time phase of a medical image), a time resolution, a matrix size, and a presence or absence of a contrast agent. In magnetic resonance imaging, the imaging parameters may further include a type of an imaging sequence, parameters such as time to repeat (TR), echo time (TE), and flip angle (FA), and a type of k-space filling trajectory. In X-ray computed tomography imaging, the imaging parameters further include X-ray conditions (tube current, tube voltage, and X-ray exposure duration, etc.), the type of scanning (non-helical scanning, helical scanning, synchronous scanning, etc.), a tilt angle, a reconstruction function, the number of views per rotation of a rotating frame, rotation speed, and the spatial resolution of the detector. In ultrasonic diagnosis, the imaging parameters further include the focal position, gain, transmission intensity, reception intensity, PRF, beam scanning scheme (sector scanning, convex scanning, linear scanning, etc.) and scanning mode (B-mode scanning, Doppler scanning, color Doppler scanning, M-mode scanning, A-mode scanning, etc.).

With the restoration capability 112, the processing circuit 11 performs restoration processing upon the raw data transmitted from the medical imaging apparatus 3 to reconstruct a medical image. The restoration processing according to the present embodiment includes restoration from raw data to raw data, restoration from raw data to image data, and restoration from image data to image data. The restoration processing from raw data defined by a certain coordinate system to two-dimensional image data or three-dimensional image data defined by a different coordinate system may also be referred to as reconstruction processing or image reconstruction processing. The restoration processing according to the present embodiment may be denoising restoration or data discrepancy feedback restoration.

Image reconstruction relating to the restoration processing according to the present embodiment may be classified into analytical image reconstruction and iterative image reconstruction. For analytical image reconstruction relating to MR image reconstruction, Fourier transform or inverse Fourier transform may be adopted. For analytical image reconstruction relating to CT image reconstruction, filtered back projection (FBP), convolution back projection (CBP), or application of such projection may be adopted. For iterative image reconstruction, expectation maximization (EM), an algebraic reconstruction technique (ART), or application of such a technique may be adopted. The processing circuit 11 that realizes the restoration capability 112 is an example of an image generating unit.

The processing circuit 11 obtains processing target data with the obtainment capability 113. According to the present embodiment, the processing target data is medical data of a processing-target subject. The processing-target medical data may be medical image data obtained through medical imaging performed upon the subject. The processing circuit 11 that realizes the obtainment capability 113 is an example of an obtainment unit.

The processing circuit 11 applies the trained model 90 to the processing target data, and thereby calculates inference data with the inference capability 114. According to the present embodiment, the processing circuit 11 applies the trained model 90 to the input medical data of the subject, and thereby generates output medical data as inference data. The output medical data may be a targeted medical image generated by executing image processing upon a medical image. The processing circuit 11 that realizes the inference capability 114 is an example of an inference unit.

With the image processing capability 115, the processing circuit 11 performs various types of image processing upon the medical image generated with the restoration capability 112, the output image generated with the inference capability 114, and the like. The processing circuit 11 may perform, for example, three-dimensional image processing such as volume rendering, surface volume rendering, pixel data projection processing, multi-planer reconstruction (MPR) processing, and curved MPR (CPR) processing. The processing circuit 11 may further perform positioning processing as image processing.

With the display control capability 116, the processing circuit 11 displays various kinds of information on the display 19. For instance, the processing circuit 11 may display a medical image generated with the restoration capability 112, an output image generated with the inference capability 114, and a medical image processed with the image processing capability 115.

Next, the inference processing operation executed in accordance with the obtainment capability 113 and inference capability 114 of the medical data processing apparatus 1 will be described. The inference processing is for applying the trained model 90 to the input medical data so that the trained model 90 can output targeted output medical data. The processing procedure of the operations indicated below is described merely as an example, and the operations may be suitably changed as needed. Omission, replacement, and addition of steps may be made to the processing procedure described below according to the embodiment.

With the inference processing, the processing circuit 11 first obtains the trained model 90 from the memory 13 with the obtainment capability 113, and obtains from the medical imaging apparatus 3 the processing-target input medical data, to which the trained model 90 is to be applied.

Next, with the inference capability 114, the processing circuit 11 applies the trained model 90 to the input medical data. The trained model 90 receives an input of the input medical data, and generates output medical data. The processing circuit 11 obtains, as an inference result, the output medical data generated by the trained model 90.

The effect of the medical data processing apparatus 1 according to the present embodiment will be described below.

The medical data processing apparatus 1 according to the present embodiment obtains processing target data, and applies the trained model 90 to the processing target data to calculate inference data. The trained model 90 includes an ensemble activation function. The ensemble activation function executes a calculation based on the activation functions and mixing coefficients respectively corresponding to the activation functions for each of the unit network structures configured to convert an input vector element to an output vector element. A unit network structure may be a node, a layer, or a channel of the neural network. A single ensemble activation function may be provided to correspond to one node, or to a plurality of nodes. In other words, a unit network structure may be a plurality of nodes, a plurality of layers, or a plurality of channels.

In particular, with the application of the ensemble activation function, a plurality of first output values can be obtained by applying a plurality of activation functions to the same input vector element, a plurality of second output values can be obtained by applying a plurality of mixing coefficients to the first output values, and an output vector element can be obtained based on the second output values. For instance, when an operation expressed by Expression (1) is executed upon the output values z1 and z2 of the activation functions A1 and A2, the ensemble activation function implements an operation based on the output values z1 and z2 and the coefficients a1, a2, and a3 to obtain output values y, and finds an output vector element y_i based on the output values y. Here, the coefficients a1, a2, and a3 correspond to the mixing coefficients, the output values z1 and z2 correspond to the first output values, and the output values y correspond to second output values.

In the medical data processing apparatus 1 according to the present embodiment, a plurality of candidate activation functions are prepared in advance, and an inference is conducted using the trained model 90 including an ensemble activation function obtained by integrating the prepared activation functions. The above configuration of the medical data processing apparatus 1 according to the present embodiment thereby realizes a higher inference performance than the configuration in which an activation function to be used is arbitrarily selected by the user.

The medical image diagnostic apparatus 9 according to the present embodiment includes the above described medical data processing apparatus 1 and a medical imaging apparatus 3 configured to perform medical imaging upon a subject. The medical data processing apparatus 1 is configured to obtain as processing target data the medical data obtained by the medical imaging apparatus 3. The medical data processing apparatus 1 is further configured to find inference data by applying the trained model 90 to the obtained medical data.

With the above configuration, the medical image diagnostic apparatus 9 according to the present embodiment can apply the trained model 90 demonstrating a high inference performance with the use of an ensemble activation function to the desired inference processing of medical data obtained by the medical image diagnostic apparatus 9. For instance, the inference performance can be improved when image processing upon a medical image obtained by the medical imaging apparatus 3 is executed using the trained model 90, in comparison with the configuration in which an activation function for the activation processing is arbitrarily selected by the user.

Next, an exemplary configuration of the model training apparatus 5 according to the present embodiment will be described. FIG. 7 is a diagram showing the configuration of the model training apparatus 5. As illustrated in FIG. 7, the model training apparatus 5 includes, as hardware resources, a processing circuit 51, a memory 53, an input interface 55, a communication interface 57, and a display 59.

The processing circuit 51 is configured to control the overall operation of the model training apparatus 5. The processing circuit 51 is a processor configured to, upon calling up and executing a program from the memory 53, implement an obtainment capability 511 and a training capability 512.

With the obtainment capability 511, the processing circuit 51 obtains training data to be used for training of the neural network. The training data includes input training data and output training data (hereinafter referred to as “labeled output data”). The processing circuit 51 that realizes the obtainment capability 511 is an example of an obtainment unit.

With the training capability 512, the processing circuit 51 trains the neural network based on the training data to generate a trained neural network. In this process, the processing circuit 51 updates a training parameter for each unit network structure and the mixing coefficients of the ensemble activation function for each unit network structure in such a manner as to minimize the loss function, which is based on a discrepancy between the output data of the neural network based on the input training data (hereinafter referred to as “inferred output data”) and the labeled output data. The processing circuit 51 that realizes the training capability 512 is an example of a training unit. The training parameter is a parameter of the composite function that defines the neural network.

The training parameter may be a weighting matrix, a bias, or the like.

The memory 53 is a storage device configured to store various types of information such as a hard disk drive (HDD), a solid state drive (SSD), and an integrated circuit. In addition to an HDD and SSD, the memory 53 may be a portable storage medium such as a compact disc (CD), a digital versatile disc (DVD), and a flash memory. The memory 53 may be a driving device for reading and writing various types of information from and to a semiconductor memory element such as a flash memory and a random access memory (RAM). The storage region of the memory 53 may be provided in the model training apparatus 5, or in an external storage device connected to the model training apparatus 5 by way of a network.

The memory 53 is configured to store programs to be executed by the processing circuit 51, various types of data to be used in the processing performed by the processing circuit 51, and the like. Such programs may include a program that is installed in advance in a computer through a network or from a non-transitory computer-readable storage medium to cause the computer to realize various functions of the processing circuit 51. The typical data discussed throughout this specification is digital data. The memory 53 is an example of a storage unit.

The memory 53 stores, for example, a model training program 50 for training the neural network. The memory 53 also temporarily stores training data that is used for training the neural network. The memory 53 further stores a plurality of activation functions that serve as candidates to be used for the ensemble activation function.

The input interface 55 receives various input operations from an operator, converts the received input operation to electric signals, and outputs the signals to the processing circuit 51. For instance, the input interface 55 receives an input of medical information and input of various command signals from the operator. The input interface 55 is realized by a mouse, a keyboard, a trackball, switch buttons, a touch screen in which a display screen and a touch pad are integrated, a non-contact input circuit adopting optical sensors, a voice input circuit, and the like to perform various kinds of processing of the processing circuit 51. The input interface 55 is connected to the processing circuit 51 so that the input operation received from the operator can be converted to an electric signal and output to the control circuit. Throughout this specification, the input interface is not limited to a physical operational component such as a mouse and a keyboard. For instance, examples of the input interface may include an electric signal processing circuit configured to receive an electric signal corresponding to an input operation from an external input device provided separately from the apparatus, and output this electric signal to the processing circuit 51. The input interface 55 is an example of an input unit.

The communication interface 57 is a network interface configured to control the communication between the medical data processing apparatus 1 and an external apparatus via a network.

The display 59 is configured to display various types of information. For instance, the display 59 outputs medical information generated by the processing circuit 51, a graphical user interface (GUI) for receiving various operations from the operator, and the like. The display 59 may be a liquid crystal display or a cathode-ray tube (CRT) display. The display 59 is an example of a display unit.

The model training processing executed by the processing circuit 51 of the model training apparatus 5 in accordance with the model training program 50 will now be described. The model training processing is for generating a trained model 90 by training a machine learning model based on the training data.

In the model training processing, the processing circuit 51 first obtains, with the obtainment capability 511, the training data including a plurality of training samples from the training data storage apparatus 7. A training sample includes a combination of input training data and labeled output data. The labeled output data is the desired data output from the neural network in response to an input of the input training data to the neural network. The labeled output data may also be referred to as “supervisory data”. The input training data and labeled output data may be medical images generated by performing imaging processing upon a patient. The input training data and labeled output data may be medical images generated by imaging a phantom.

Next, with the training capability 512, the processing circuit 51 generates inferred output data in accordance with the forward propagation of the neural network based on the input training data. In the first forward propagation, the parameter of the neural network is set to an initial value. With the training capability 512, the processing circuit 51 calculates the discrepancy between the generated inferred output data and the input labeled output data. Next, with the training capability 512, the processing circuit 51 calculates a gradient vector in accordance with the reverse propagation of the neural network based on the calculated discrepancy. Thereafter, with training capability 512, the processing circuit 51 updates the parameters of the entire neural network including the mixing coefficients, based on the calculated gradient vector.

For instance, when the ensemble activation function is a function that performs an operation expressed in the aforementioned Expression (1) for integrating the activation functions A1 and A2, the training parameter is updated, and the coefficients a1 to a3 in Expression (1) are also updated as mixing coefficients through the training of the neural network.

When the ensemble activation function is a function that performs an operation expressed in the aforementioned Expression (2) for integrating the activation functions A1 and A2, the training parameter is updated by training the neural network, and the coefficients a1 to a6 in Expression (2) are updated as mixing coefficients.

The processing circuit 51 determines whether or not a termination condition is satisfied. The termination condition may be set, for example, to the number of repetitions reaching a preset number, or the termination condition may be set to the gradient vector falling below a threshold value. When the termination condition is not met, the processing circuit 51 repeats the processing, using the same training sample or a different training sample. When the termination condition is met, the processing circuit 51 outputs the updated neural network as a trained model 90. The trained model 90 is stored in the memory 13 of the medical data processing apparatus 1.

The model training processing by the model training apparatus 5 according to the present embodiment is as described above. The above flow of the learning process is described as an example, and the present embodiment is not limited thereto.

The model training program 50 according to the present embodiment causes the model training apparatus 5 to implement the training capability 512, as described above. With the training capability 512, inferred output data is generated by applying a neural network to the input training data, where the neural network includes an input layer to which input training data is input, an output layer from which output data corresponding to the input training data is output, and at least one intermediate layer provided between the input layer and output layer. With the training capability 512, the parameter of the neural network having mixing coefficients is updated in such a manner that the inferred output data and labeled output data approximate each other.

The model training apparatus 5 according to the present embodiment is capable of acquiring the training data, which includes the input training data and output training data, training the neural network based on the training data, and generating a trained neural network. The neural network includes unit network structures with a training parameter, each unit network structure converting an input vector element to an output vector element. The neural network further includes an ensemble activation function, which implements an operation for each of the unit network structures based on the activation functions and the mixing coefficients respectively corresponding to the activation functions. The model training apparatus 5 is capable of updating the training parameter and mixing coefficients in such a manner as to minimize the discrepancy between the output data of the neural network based on the input training data and the output training data.

With the above configuration, the function for implementing the activation processing can be generated through machine learning according to the present embodiment. In this manner, the optimal parameter can be set as a mixing coefficient of the ensemble activation function, as a result of which an inference apparatus with improved inference performance can be realized.

### (First Modification Example)

The first modification example of the embodiment will be described. This modification example is based on a modification described below to the configuration of the embodiment. The portion of the structure, operations and effects that is similar to that of the embodiment will be omitted from the description.

In this modification example, a neural network that outputs a plurality of output datasets in response to a single input dataset will be described. The neural network is configured to receive, for example, a medical image as input data and output a plurality of medical images subjected to different types of image processing.

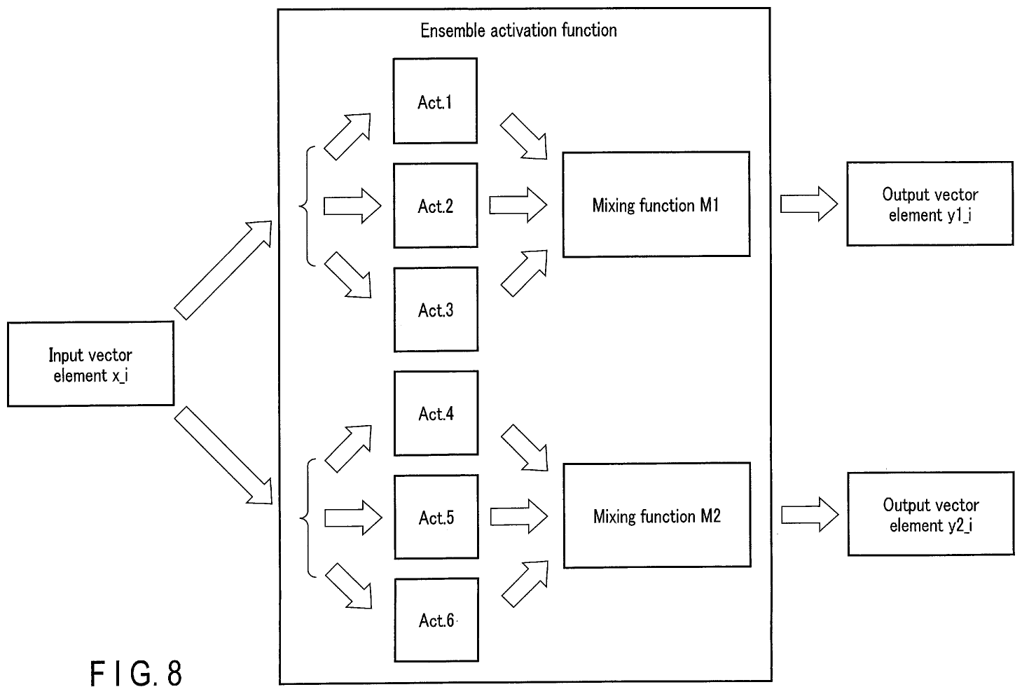

FIG. 8 is a diagram schematically showing a calculation performed by the ensemble activation function according to the present modification example. In the example of FIG. 8, the ensemble activation function is constituted by six activation functions A1 to A6 and two mixing functions M1 and M2. The activation functions A1 to A6 are denoted as “Act.1” to “Act.6” in FIG. 8.

The activation functions A1 to A3 differ from each other in types of activation functions. The activation functions A4 to A6 also differ from each other in types of activation functions. The types of activation functions A1 to A3 and the types of activation functions A4 to A6 may match, or may differ from each other.

The input vector element x_i that has been input into the ensemble activation function is inserted into each of the activation functions A1 to A6. The activation functions A1 to A6 respectively receive the values x of the input vector element x_i, and output the results of the application of the activation functions to the values x, as output values z1 to z6.

The mixing function M1 integrates the three output values z1 to z3 obtained from the activation functions A1 to A3 in accordance with the mixing coefficients to find an output value y1. The mixing function M2 integrates the three output values z4 to z6 obtained from the activation functions A4 to A6 in accordance with the mixing coefficients to find an output value y2.

The above activation functions A1 to A6 and mixing functions M1 and M2 are sequentially applied to the values x included in the input vector element x_i, as a result of which the output values y1 and y2 corresponding to the respective values x included in the input vector element x_i are output. The ensemble activation function generates an output vector element y1_i including the output value y1 and an output vector element y2_i including the output value y2, and outputs them as the outputs of the ensemble activation function.

Furthermore, the neural network for outputting output datasets in response to a single input dataset may be configured to apply activation functions of a plurality of types to the input dataset, use the outputs of the activation functions as inputs of the next operation for a plurality of channels, and repeat the convolution processing and activation processing to output the output datasets. For instance, when there are three activation functions and ten channels of input data to be input to these activation functions, there will be thirty channels for input in the next convolution processing. In this case, the results of the ensemble operation in the ensemble activation function serve as some of the coefficients of the next convolution processing, producing the same effects as in the aforementioned embodiment.

### (Second Modification Example)

The second modification example of the embodiment will be described. This modification example is based on a modification described below to the configuration of the embodiment. The portion of the structure, operation and effects that is similar to that of the embodiment will be omitted from the description.

The ensemble activation function according to this modification example is configured to apply a plurality of mixing coefficients to the same input vector element to find the first output values, apply a plurality of activation functions to the first output values to find the second output values, and obtain an output vector element based on the second output values.

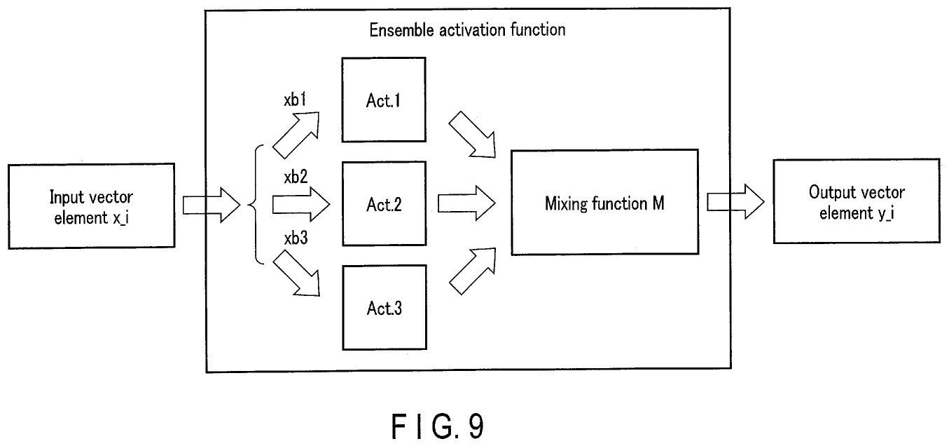

FIG. 9 is a diagram schematically showing a calculation performed by the ensemble activation function according to the present modification example. Here, an example of the ensemble activation function by which activation functions A1, A2, and A3 are integrated will be discussed. The ensemble activation function multiplies the input value x input to the activation function A1 by a coefficient b1 to find an output value z′1 (=b1·x), multiplies the input value x input to the activation function A2 by a coefficient b2 to find an output value z′2 (=b2·x), and multiplies the input value x input to the activation function A3 by a coefficient b3 to find an output value z′3 (=b3·x). Each of the output values z′1 to z′3 is the first output value. Each of the coefficients b1 to b3 is a mixing coefficient.

Thereafter, the ensemble activation function applies the activation function A1 to the output value z′1, the activation function A2 to the output value z′2, and the activation function A3 to the output value z′3. Then, the ensemble activation function applies the mixing function M to the output value z1 of the activation function A1, to the output value z2 of the activation function A2, and to the output value z3 of the activation function A3 to find the output value y. Each of the output values z1, z2, and z3 is the second output value. The mixing function M may be a linear function or a quadratic function having output values z1, z2, and z3 as variables, or may be any function in the form of a polynomial having a dimensionality of 3 or larger.

### (Third Modification)

The third modification example of the embodiment will be described. This modification example is based on a modification described below to the configuration of the embodiment. The portion of the structure, operation and effects that is similar to that of the embodiment will be omitted from the description.

The ensemble activation function according to this modification example finds an output vector element by applying to an input vector element a function reconstructed based on a plurality of mixing coefficients and a plurality of activation functions.

Here, an example of the ensemble activation function by which activation functions A1 and A2 are integrated will be discussed. The ensemble activation function executes an operation using a function reconstructed in advance by multiplying the activation function A1 by a coefficient c1 and a function reconstructed in advance by multiplying the activation function A2 by a coefficient c2. Each of the coefficients c1 and c2 is a mixing coefficient.

The ensemble activation function applies each of the reconstructed activation functions A1 and A2 to the input value x, and applies the aforementioned mixing function M to the output value z1 of the reconstructed activation function A1 and to the output value z2 of the reconstructed activation function A2 to find an output value y. The mixing function M may be a linear function or a quadratic function having output values z1 and z2 as variables, or a polynomial having a dimensionality of 3 or larger.

### (Fourth Modification Example)

The fourth modification example of the embodiment will be described. This modification example is based on a modification described below to the configuration of the embodiment. The portion of the structure, operation and effects that is similar to that of the embodiment will be omitted from the description.

In the neural network according to this modification example, a regularization method is incorporated to mitigate overfitting.

As a regularization method, all mixing coefficients may have the same value. For instance, when the ensemble activation function employs the above Expression (1) to integrate the output values z1 and z2 of the activation functions A1 and A2, overfitting can be mitigated by assigning the same value to the coefficients a1 to a3 of all of the ensemble activation functions in the respective unit network structures.

The ensemble activation functions in a network structure may be sorted into groups so that the mixing coefficients of all the ensemble activation functions in the same group may be determined to have the same value.

Alternatively, among the ensemble activation functions in the neural network, the mixing coefficients corresponding to the same activation function may be determined to have the same value. In this case, the mixing coefficients (e.g., a1 in Expression (1)) corresponding to the same activation function type (e.g., activation function A1) will be determined to have the same value in all the ensemble activation functions.

According to the present embodiment, the entire neural network is trained so that an ensemble activation function is generated for each unit network structure. The resultant ensemble activation functions may differ or may be the same function among the unit network structures.

Other possible regularization methods include adding a cost function whose value decreases with a smaller disparity between the mixing coefficients to a loss function based on the disparity between the inferred output data and the input labeled output data. The cost function may be, for example, a sum of squares of the mixing coefficients. With such a cost function, when updating the training parameters and mixing coefficients in such a manner as to minimize the disparity between the inferred output data and the labeled output data, the parameters are optimized in such a manner as to minimize the disparity between the mixing coefficients, thereby suppressing the overfitting.

As another possible regularization method, transfer learning using training data outside the objective may be applied. With this method, training of a neural network using normal training data is performed by adopting, as initial values, the training parameters and mixing coefficients of a trained neural network generated by training the neural network based on training data outside the objective. For instance, if the normal training data is medical image data, the neural network may be trained by adopting non-targeted training data such as image data in a general field other than a medical field to generate a trained model. Then, the neural network is trained with the training parameters and mixing coefficients of the generated trained model as initial values and with medical image data as training data. In this manner, overfitting that would lower the generalization capability can be suppressed, and the inference performance can be improved.

Other regularization methods include data augmentation, addition of an L2 regularization term corresponding to a training parameter and/or a mixing coefficient to a loss function, and a sparse regularization performed by adding the L2 regularization term corresponding to the training parameter and/or mixing coefficient to a loss function. Furthermore, L2 regularization or L1 regularization upon the disparities of the training parameters and mixing coefficients after implementation of the aforementioned transfer learning may be adopted.

The regularization, however, should not be limited to the above methods, and various regularization methods that are commonly used may be adopted. Some of the above methods may be combined in use.

### (Other Modification Examples)

The neural network according to the present embodiment is applicable to a complex network. The neural network according to the present embodiment is also applicable to a neural network without using a bias. For instance, in a complex network for denoising complex images such as MRI images without a bias, an ensemble activation function according to the present embodiment may be adopted in place of an activation function.

The neural network according to the present embodiment is effective for an inference apparatus adopting complex data as processing target data. Which activation function or which neural network to use may be determined in accordance with whether or not the obtained processing target data is complex data. For instance, inference processing may be implemented with a neural network using an ensemble activation function according to the present embodiment only when the obtained processing target data is MRI image data obtained by a magnetic resonance imaging apparatus or echo data obtained by an ultrasonic diagnostic apparatus.

The present embodiment has dealt with a neural network for medical data as an example. The ensemble activation function according to the present embodiment, however, may be applicable to a neural network that executes image recognition upon general images. For instance, such a neural network may be a DNN that outputs the subject of an image being a “cat”, “dog”, “horse”, or “cow”. To train this neural network, images bearing some animals are used as input training data, and a one-hot vector indicating a type of animal such as a “cat”, “dog”, “horse”, or “cow” is used as labeled output data.

According to at least one of the above embodiments, the inference performance of a trained neural network using an activation function can be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An inference apparatus comprising a processing circuit configured to:
obtain processing target data; and
calculate inference data by applying a trained neural network to the processing target data,
wherein the trained neural network includes an ensemble activation function for each of a plurality of unit network structures configured to convert an input vector element to an output vector element, the ensemble activation function being configured to execute a calculation based on a plurality of activation functions and a plurality of mixing coefficients respectively corresponding to the activation functions.

2. The inference apparatus according to claim 1, wherein
the ensemble activation function calculates a plurality of first output values by applying the activation functions to the same input vector element, calculates a plurality of second output values by applying the mixing coefficients to the first output values, and calculates the output vector element based on the second output values.

3. The inference apparatus according to claim 1, wherein
the ensemble activation function calculates a plurality of first output values by applying the mixing coefficients to the same input vector element, calculates a plurality of second output values by applying the activation functions to the first output values, and calculates the output vector element based on the second output values.

4. The inference apparatus according to claim 1, wherein
the ensemble activation function calculates the output vector element by applying to the input vector element a function reconstructed based on the mixing coefficients and the activation functions.

5. A medical image diagnostic apparatus comprising:
the inference apparatus according to claim 1; and
a medical imaging apparatus configured to perform medical imaging upon a subject,
wherein the processing circuit obtains, as the processing target data, medical data obtained by the medical imaging apparatus.

6. An inference method, comprising:
obtaining processing target data; and
calculating inference data by applying a trained neural network to the processing target data,
wherein the trained neural network includes an ensemble activation function for each of a plurality of unit network structures configured to convert an input vector element to an output vector element, the ensemble activation function being configured to execute a calculation based on a plurality of activation functions and a plurality of mixing coefficients respectively corresponding to the activation functions.

7. A method for generating a trained neural network, comprising:
obtaining training data that includes input training data and output training data; and
learning that includes training a neural network based on the training data and generating a trained neural network,
wherein the neural network includes:
a plurality of unit network structures with a training parameter attached, the unit network structures being configured to convert an input vector element to an output vector element; and
an ensemble activation function configured to execute a calculation for each of the unit network structures based on a plurality of activation functions and a plurality of mixing coefficients corresponding to the activation functions, and
the learning includes updating the training parameter and the mixing coefficients based on the input training data in such a manner as to minimize a discrepancy between the output data of the neural network and the output training data.

8. The method for generating the trained neural network according to claim 7, wherein
the learning includes determining mixing coefficients corresponding to the same activation function to have a same value in the ensemble activation functions included in the neural network.

9. The method for generating the trained neural network according to claim 7, wherein
the learning includes adding a cost function to a loss function, where a value of the cost function decreases as a discrepancy between the mixing coefficients is reduced.

10. The method for generating the trained neural network according to claim 7, wherein
the learning includes training the neural network based on the training data, using a training parameter and a mixing coefficient of a trained neural network as initial values, the trained neural network being generated through training the neural network based on training data outside an objective.
